(19) United States
(12) Patent Application Publication
(10) Pub. No.: US 2022/0061681 A1
(43) Pub. Date: Mar. 3, 2022

(54) SYSTEMS AND METHODS FOR HUMAN BEHAVIOR MANAGEMENT IN MEDICAL AND BEHAVIORAL TREATMENT

(71) Applicant: Ken Hopper, Fort Worth, TX (US)

(72) Inventor: Ken Hopper, Fort Worth, TX (US)

(21) Appl. No.: 17/465,437

(22) Filed: Sep. 2, 2021

Related U.S. Application Data
(60) Provisional application No. 63/073,571, filed on Sep. 2, 2020.

(51) Int. Cl. A61B 5/0205; A61B 5/00; A61B 5/16; G06N 20/00; G16H 20/13

(52) U.S. Cl. CPC A61B 5/02055; A61B 5/0022; A61B 5/165; A61B 5/746; G06N 20/00; G16H 20/13; A61B 5/6813; A61B 5/7267

(57) ABSTRACT

Systems and methods for operating a system to facilitate behavior management of an individual. The methods comprise: receiving, by a computing device, sensor data from at least one sensor device in proximity to the individual; processing, by the computing device, the sensor data to detect when at least a threshold probability of a given mindfulness state for the individual is indicated by at least one of a location, an activity and a physical characteristic of the individual; and causing, by the computing device, a mindfulness risk remediation event to occur when the threshold probability of the given mindfulness state for the individual is detected.

### CROSS-REFERENCE TO RELATED APPLICATION

The present application claims priority to U.S. Provisional Patent Application No. 63/073,571, filed Sep. 2, 2020, entitled “SYSTEMS AND METHODS FOR HUMAN BEHAVIOR MANAGEMENT IN MEDICAL AND BEHAVIORAL TREATMENT,” which is incorporated herein by reference in its entirety.

### FIELD OF THE INVENTION

The present document relates to computing systems. More particularly, the present document relates to systems and methods for human behavior management in medical and behavioral treatment.

### BACKGROUND

The transition of American Healthcare from an Acute Care orientation to a Chronic Condition Management environment has been catalyzed by multiple forces. In the center are diagnoses such as obesity, diabetes, chronic pain, heart disease, and stress (or, bonafide Depression). These conditions are not isolated illnesses, but rather build and “spiral downward” with each other.

There exists many different barriers or obstacles in the present system of medical care. In terms of medical professionals, the idea of accepting and incorporating a “best practice” method for handling their actions and approach to treatment is not readily accepted. Justifications are made to support the reluctance to accept “best practices”.

Beyond just the medical professionals, the structure of the care industry can be difficult to navigate especially for patients. Each specialty can be individualized with a lack of continuity from one specialty to another. Specialists operate differently and separately from general care physicians. Methodology in how to process and handle data consistently hinders the care process.

Furthermore, too often, both patients and professionals consider the patient as a bystander rather than as a participating partner in the patient's own care and treatment. Habits need to be made or changed and “buy-in” needs to occur in full by the patient and the professionals involved in the patient's care. The goals or directions of the professional and the patient often are not synchronized, which leads to inefficient and even ineffective or less optimum treatment results.

### SUMMARY

The present document concerns implementing systems and methods for operating a system to facilitate behavior management of an individual. The methods comprise: receiving, by a computing device, sensor data from at least one sensor device in proximity to the individual (e.g., within 0-30 feet of the individual); processing, by the computing device (e.g., using a machine learning model and/or algorithm), the sensor data to detect when at least a threshold probability of a given mindfulness state for the individual is indicated by a location, an activity and/or a physical characteristic of the individual; and causing, by the computing device, a mindfulness risk remediation event to occur when the threshold probability of the given mindfulness state for the individual is detected. The threshold probability can be detected, for example, when there is at least a 75% probability or likelihood that the individual currently has or will have a mental health episode (e.g., experience depression), have a particular thought or feeling, and/or perform a given harmful action to himself(herself) or another person.

The computing device may include a medication dispenser which may or may not be wearable. The medication dispenser may be configured to automatically dispense a given quantity of medication into the individual when actuated. The mindfulness risk remediation event can include, but is not limited to, dispensing the given amount of medication to the individual, and/or generating and sending a notification notifying one or more parties of the threshold probability of the given mindfulness state has been determined for the individual.

In some scenarios, the methods also comprise performing operations by the computing device and/or another computing device to automatically schedule a medical examination for the individual when the threshold probability of the given mindfulness state is detected.

The sensor device(s) can include(s), but is(are) not limited to, camera(s), Global Positioning System (“GPS”) device(s), fitness tracker(s), thermometer(s), smart phone(s), and/or health monitor(s). The sensor device(s) may be wearable sensor devices configured to be worn by the individual. The sensor device(s) may be configured to sense physical movements, object motions, heart rate, temperature, blood pressure, breathing patterns and/or sleep patterns.

As noted above, a machine learning model and/or algorithm can be used to determine whether a location, an activity, or a physical characteristic of the individual indicates at least a threshold probability of a given mindfulness state for the individual. The machine learning model and/or algorithm can be trained using a training data set comprising sensor data associated with the individual and/or other individual(s).

The present document also concerns systems for facilitating behavior management of an individual. The systems include sensor device(s) that is(are) located in proximity to the individual and configured to collect sensor data. The system further includes a first computing device, including a processor and a memory, configured to store programming instructions that, when executed by the processor, cause the first computing device to receive the sensor data from the sensor device(s). The sensor data is processed to detect when a location, an activity, or a physical characteristic of the individual indicates at least a threshold probability of a given mindfulness state for the individual. A mindfulness risk remediation event is caused to occur when the threshold probability of the given mindfulness state for the individual is detected.

The first computing device can include, but is not limited to, a medication dispenser configured to automatically dispense a given quantity of medication into the individual when actuated. The medication dispenser may comprise a fluid chamber filled with liquid medicine, a valve for allowing liquid medicine to flow out of the fluid chamber, and/or a nozzle or syringe for dispensing the liquid medicine onto the individual's skin or into the individual's bloodstream. The mindfulness risk remediation event can include, but is not limited to, actuating the medication dispenser for dispensing the given amount of medication, and/or generating and sending a notification notifying one or more parties of the threshold probability of the given mindfulness state associated with the individual.

The system may also comprise a second computing device. The first and/or second computing devices can be configured to facilitate an automatic scheduling of a medical examination for the user when the threshold probability of the given mindfulness state for the individual is detected.

The sensor device(s) can include, but are not limited to, camera(s), GPS device(s), fitness tracker(s), thermometer(s), smart phone(s), and/or health monitor(s). The sensor device(s) is(are) wearable sensor(s) configured to be worn by the individual. The sensor device(s) may be configured to sense physical movements or motions, heart rate, temperature, blood pressure, breathing patterns, and/or sleep patterns.

### BRIEF DESCRIPTION OF THE DRAWINGS

The present solution will be described with reference to the following drawing figures, in which like numerals represent like items throughout the figures.

FIG. 1 provides an illustration of a system.

FIG. 2 provides an illustration of a computing device.

FIG. 3 provides an illustration of a mobile communication device.

FIG. 4 provides an illustration of another illustrative system.

FIG. 5 provides a chart of the system shown in FIG. 1 and the system shown in FIG. 4.

FIG. 6 provides a flowchart of an illustrative method that is useful for understanding operations of the system of FIG. 1 and the system of FIG. 4.

FIG. 7 provides a chart of grading rules used in the system of FIG. 1 and the system of FIG. 4.

FIGS. 8A-8C (collectively referred to as “FIG. 8”) provides a flow diagram of an illustrative method for operating a behavior management system.

FIG. 9 provides a method for operating a system to facilitate behavior management of individual(s).

### DETAILED DESCRIPTION

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms to describe a spatial relationship between various components or to describe the spatial orientation of aspects of such components should be understood to describe a relative relationship between the components or a spatial orientation of aspects of such components, respectively, as the embodiments described herein may be oriented in any desired direction.

The embodiments and method will be understood, both as to its structure and operation, from the accompanying drawings, taken in conjunction with the accompanying description. Several embodiments of the assembly may be presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless otherwise described.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” means “including, but not limited to.” Definitions for additional terms that are relevant to this document are as follows. The term mindfulness risk as used herein refers to a risk of potential direct adverse effects caused by the activities performed by an individual. Such adverse effects may include, but are not limited to, psychosis, mania, depersonalization, anxiety, panic, memory re-experiencing, and other forms of clinical deterioration.

An “electronic device” or a “computing device” refers to a device that includes a processor and memory. Each device may have its own processor and/or memory, or the processor and/or memory may be shared with other devices as in a virtual machine or container arrangement. The memory will contain or receive programming instructions that, when executed by the processor, cause the electronic device to perform one or more operations according to the programming instructions.

The terms “memory,” “memory device,” “data store,” “data storage facility” and the like each refer to a non-transitory device on which computer-readable data, programming instructions or both are stored. Except where specifically stated otherwise, the terms “memory,” “memory device,” “data store,” “data storage facility” and the like are intended to include single device embodiments, embodiments in which multiple memory devices together or collectively store a set of data or instructions, as well as individual sectors within such devices.

The terms “processor” and “processing device” refer to a hardware component of an electronic device that is configured to execute programming instructions. Except where specifically stated otherwise, the singular term “processor” or “processing device” is intended to include both single-processing device embodiments and embodiments in which multiple processing devices together or collectively perform a process.

Although strides have been made with respect to improvements in the level of care afforded to patients and provided by medical professionals, considerable shortcomings remain. It is desired that a system works with both patients and professionals in a manner that is proactive in empowering them to work together to facilitate a high standard of care. The best way to address any of these conditions is to prevent them from taking root in chronicity. The optimal way to handle these conditions, once discovered, is through restorative actions back towards health and wellness. These changes in course are demonstrably possible, but not easy.

A goal of the present solution is the preparation for, or anticipation of, upcoming actions by medical professionals and patients. By being preemptive and prepared, no longer is the medical provider stuck in a patient-chasing or after-the-fact reactionary method, as seen with most clinical operations. Medical providers and patients are empowered due to the orchestrated tools (i.e., pathways) at the point of service. An order sheet or activity flow sheet may be associated with each pathway. The order sheet or activity flow sheet is used to capture information and guide the actions of the medical provider or support personnel. This helps to maintain complete transparency in a medical clinic. This may also allow for reduced operational staff, thereby further decreasing overhead expenses.

Another goal of the present solution is the integration of both behavioral/cultural change in patients and professionals along with the enhancements of the physical processes of medical treatment methods. Both patients and professionals have a responsibility for the overall effectiveness of care received. By encouraging a team approach between professionals and patients, a higher level of care may be achieved. Implementing systems provide functions and features to generate better acceptance of the care prescribed and help them to buy-in to the treatment program. The implementing systems also help to facilitate appropriate mapped processes as they relate to a treatment and actions of the professionals. One or more software programs may be employed by the implementing systems to permit better clarity, streamline actions, enhance integration, and define roles within the practice. Also, a set of best practices or predefined steps are encouraged for use by the implementing systems to streamline and enhance the quality of patient care.

Illustrative Systems

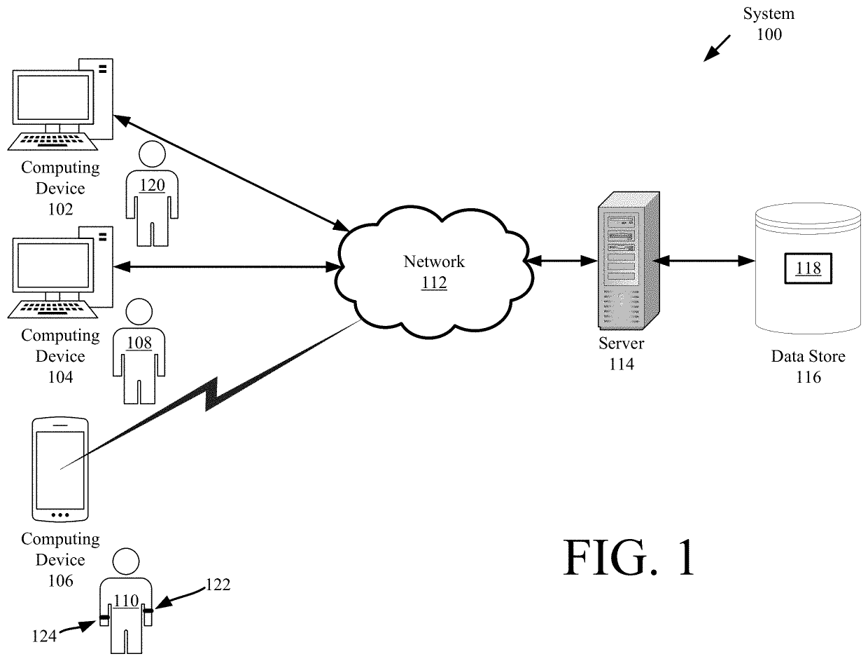

Referring now to FIG. 1, there is provided an illustration of a system 100 for predicting and proactively guiding human behavior, as applied to both professionals and patients. System 100 is used to ensure both professionals and patients together identify and address the needs of the patients. System 100 operates to empower and encourage patients in performing the needed and requested treatments and actions prescribed by the professionals. At the same time, system 100 assists the professionals in the identification of patient needs, classification of patient conditions, and coordination of professionals in providing a higher standard of care. Overall, system 100 is a unique system configured to streamline a patient's movement through a treatment process. System 100 is ideally suited for use with multiple disciplines and occupational practices. System 100 will be described herein in relation to a medical practice (e.g., a psychiatric practice). The present solution is not limited in this regard.

A goal or intent of system 100 is to place the medical provider (i.e., doctor or other medical professional) on common ground with that of the patient and other medical providers. Differing points of view between medical providers and patients can sometimes hinder the blended and uniform views. Patients are to be encouraged to participate and keep along the prescribed path for wellness. Medical providers adopt a streamlined and efficient practice of care through use of system 100. System 100 tracks and directs sequential actions of the medical provider in an effort to maintain a high level of care.

Areas of focus are many and can include the point of service or visit of the patient, proactive planning on the part of the medical provider, and cognitive behavioral directions, to name a few. System 100 is configured to assist in point of service care by capitalizing on the visit transaction as a place to focus energy and resources. Both parties recognize that arranging consults and reducing system barriers can produce better results. Additionally, system 100 may include the incorporation of motivational and cognitive-behavioral focal discussions between the patients and medical providers with highly specific and agreed upon actions to further enhance the care process. By way of example, patients who are screened for the ability to participate in a cognitive-behavioral change program may be issued access to a digital application.

According to various embodiments, the digital application is applied both to a user device (e.g., a smart phone, tablet, laptop, desktop, an application-dedicated device, and/or other suitable electronic device) and a device (e.g., a smart phone, tablet, laptop, desktop, an application-dedicated device, and/or other suitable electronic device) for telemedicine visit use. The digital application is configured to enable the user (e.g., a patient) to receive and/or send messages. According to various embodiments, the digital application is configured to notify (e.g., via sound, visual effects, and/or other suitable means) the user and/or a third party when a message has been sent and/or received. According to various embodiments, the messages are configured to provide the user with health-related instructions. As the user (e.g., patient) learns more about their motivators and blocks, individually tailored solutions may be embedded into customizable scripts in the application. According to various embodiments, medical professionals may use a mirror application. The application may provide (via, e.g., visual, audio, and/or other suitable means) guidance and/or instructions for aiding a user in preparation for one or more clinical visits

According to various embodiments, the application links biometric data for the user to proactive awareness tools and solutions for the user, including medication dosing. Physiologic and medical principles behind certain biometric associations are well-known. For example, blood pressure and pulse (heart rate) are known to elevate (“spike”) in stressful, anxiety-provoking contexts. These readings can be used as signals for interventions and can, over time, be controlled simply through the cognitive-behavioral arm of interventions.

According to various embodiments, using the application, users (e.g., patients) are guided to explore the likely circumstances in which the users may be stressed. Experiential therapy is applied to understand the effectiveness of various contextual cognitive-behavioral interventions for one or more different circumstances. Additionally, using the application, the user may be provided tasks or insight in order to be trained in a near-event dosing of a Beta-Blocker medication (such as propranolol) as an escalated intervention option. According to various embodiments, cognitive-behavioral and/or medication solutions are tested in scripted scenarios for their efficacy and for any unexpected problems. According to various embodiments, the resulting biometric data (e.g., blood pressure and heart rate elevation ranges) and gradated cognitive-behavioral and medication interventions are entered by a third party (e.g., medical professions, clinic program staff, etc.) into a templated query that aligns patient and clinic action via mirrored configurations within the application.

According to various embodiments, based on biometric range parameters, customized and gradated cognitive-behavioral solutions, and gradated medication dosing instructions, the patient can be prompted as biometric data is downloaded wired and/or wireless means (e.g., from one or more sensors such as, e.g., wearable blood pressure/heart rate monitors, etc.). according to various embodiments, the application may run without outside or direct clinical intervention unless, e.g., the patient requests the intervention or biometrics are significantly out of range. It is noted, however, that, in other embodiments, the application may run within outside or direct clinical intervention. Through shared patient-clinic programming, all data is captured using the application. The transcript of such becomes a self-improvement and learning loop for future clinical sessions and program advancement. An accelerated track of self-awareness, self-efficacy, and proactive perspective is the targeted result.

System 100 is also focused on proactive planning and assisting the medical provider in being efficiently prepared for visits by the patient. System 100 implements a core registry process which facilitates relatively quick input/data analysis (e.g., inputs and data are analyzed in a matter of hours). The results of the input/data analysis are then applied to optimized best practices in the medical clinic. The review and subsequent action that results from the data analysis can occur outside the visit.

Life lessons and cognitive behavioral encouragements are provided through system 100 to the patients and/or medical providers. Mundane clinical and life transactional events provide an opportunity to create commitment, reduce anxiety and emotional barriers, and demonstrate productive problem-solving. Historically, psychotherapy has used the therapist-patient relationship as a metaphor for other relationships and transactional styles. System 100 may incorporate “pop-up” talking points and scripts for image-creating storytelling helps the provider in-the-moment of conversation.

As an optional feature of system 100, a behavioral program may be incorporated therein to encourage and develop greater skill and control in one's life. The behavioral program may be geared to the patients and/or medical providers. System 100 may enhance engagement and persistency in the therapeutic relationship. The stages of immersion, learning, intern, and mastery are defined by milestone achievements. The stages signify progressive levels of cognitive-behavioral empowerment required for optimized self-management.

In some scenarios, system 100 actualizes the coordination of mental health care with the patient's medical care. In other words, system 100 provides a way for the practitioner to put into practice what the medical and mental health communities have been discussing for a while through a set of program rules, directions, or systematic steps. Some key functions of system 100 is the ability to move beyond the mere reactive nature of care where treatment is only prescribed after an issue has arisen, and reach a proactive method of care where actions are prescribed and followed to head off issues that may arise. Furthermore, system 100 is configured to assist in coordinating multiple medical professionals through a plurality of disciplines quickly to change from care through an individual provider to that of a collective effort providing best practices across the field of medicine.

A further function of system 100 includes the ability of the system to track and monitor the condition of the patient and the performance of the patient with respect to assigned tasks and assignments from the professional. System 100 allows professionals to better view the patient as a whole, with respect to each patient's unique set of conditions, and prescribe appropriate actions to better help the patient.

This monitoring also permits system 100 to automatically process data with vetted escalation and alert paths for identifying areas of exception in the treatment process. The goal is to bring forward in a coordinated effort the expertise of multiple disciplines and professionals to streamline care, accurately monitor patients, and provide a proven structure of communication to effectuate change. System 100 comprises multiple components containing a degree of software and hardware used and programmed in a manner to facilitate such functions.

As shown in FIG. 1, system 100 comprises computing device(s) 102, 104, 106 communicatively coupled to server(s) 114 via a network 112. Network 112 can include, but is not limited to, the Internet, an Intranet, a WiFi network, and/or a cellular network. Computing devices 102, 104 are used by medical professional(s) 108, 120, while computing device 106 is used by a patient 110. The computing devices 102, 104, 106 facilitate the collection of information associated with the patient's health and medical treatment by system 100. This information is stored in a data store 116 (e.g., a database) as data 118. The data 118 is accessed and processed by server 114 and/or computing devices 102, 104, 106 to allow medical professionals 108, 120 of multiple disciplines to streamline care of the patient 110, accurately monitor the health of the patient 110, and more effectively communicate with the patient 110 to effectuate change in the patient's behavior (e.g., through the wearable sensor/device 124 of FIG. 1 and/or other devices in the patient's possession).

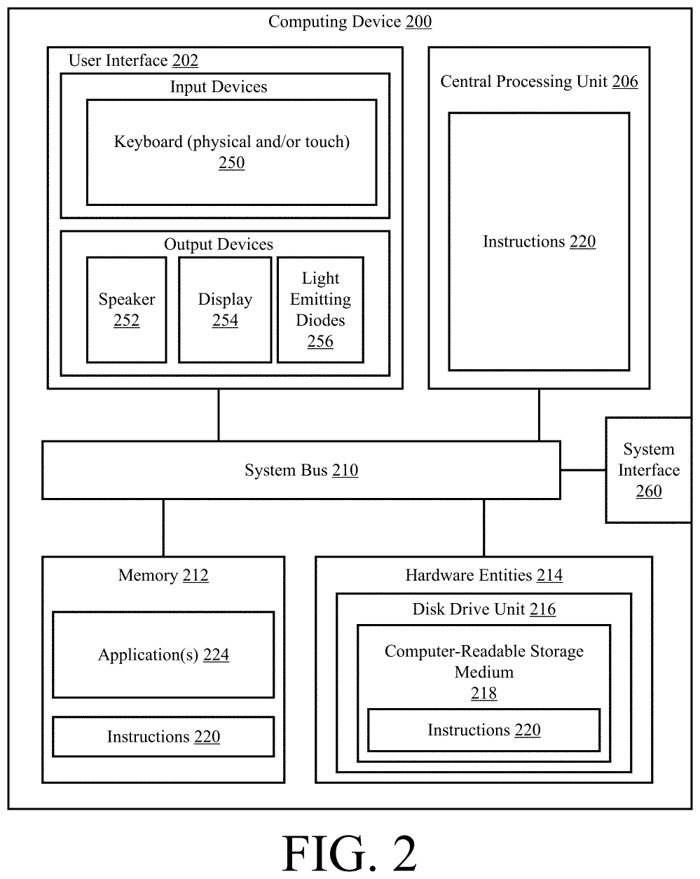

Referring now to FIG. 2, there is provided an illustration of an illustrative architecture for a computing device 200. Computing devices 102, 104 of FIG. 1 and server 114 of FIG. 1 are the same as or similar to computing device 300. As such, the discussion of computing device 200 is sufficient for understanding computing device 220 102, 104 of FIG. 1 and server 114 of FIG. 1.

Computing device 200 may include more or less components than those shown in FIG. 2. However, the components shown are sufficient to disclose an illustrative solution implementing the present solution. The hardware architecture of FIG. 2 represents one implementation of a representative computing device configured to facilitate behavior management, as described herein. As such, the computing device 200 of FIG. 2 implements at least a portion of the method(s) described herein.

Some or all components of the computing device 200 can be implemented as hardware, software and/or a combination of hardware and software. The hardware includes, but is not limited to, one or more electronic circuits. The electronic circuits can include, but are not limited to, passive components (e.g., resistors and capacitors) and/or active components (e.g., amplifiers and/or microprocessors). The passive and/or active components can be adapted to, arranged to and/or programmed to perform one or more of the methodologies, procedures, or functions described herein.

As shown in FIG. 2, the computing device 200 comprises a user interface 202, a Central Processing Unit (“CPU”) 206, a system bus 210, a memory 212 connected to and accessible by other portions of computing device 200 through system bus 210, a system interface 260, and hardware entities 214 connected to system bus 210. The user interface can include input devices and output devices, which facilitate user-software interactions for controlling operations of the computing device 200. The input devices include, but are not limited to, a physical and/or touch keyboard 250. The input devices can be connected to the computing device 200 via a wired or wireless connection (e.g., a Bluetooth® connection). The output devices include, but are not limited to, a speaker 252, a display 254, and/or light emitting diodes 256. System interface 260 is configured to facilitate wired or wireless communications to and from external devices (e.g., network nodes such as access points, wearable sensor/device 124 of FIG. 1, etc.). The communications can include, but are not limited to, commands for controlling operations of the external devices and/or reminders/stimulus for individual in possession of the external devices.

At least some of the hardware entities 214 perform actions involving access to and use of memory 212, which can be a Random Access Memory (“RAM”), a disk drive, flash memory, a Compact Disc Read Only Memory (“CD-ROM”) and/or another hardware device that is capable of storing instructions and data. Hardware entities 214 can include a disk drive unit 216 comprising a computer-readable storage medium 218 on which is stored one or more sets of instructions 220 (e.g., software code) configured to implement one or more of the methodologies, procedures, or functions described herein. The instructions 220 can also reside, completely or at least partially, within the memory 212 and/or within the CPU 206 during execution thereof by the computing device 200. The memory 212 and the CPU 206 also can constitute machine-readable media. The term “machine-readable media”, as used here, refers to a single medium or multiple media (e.g., a centralized or distributed database, and/or associated caches and servers) that store the one or more sets of instructions 220. The term “machine-readable media”, as used here, also refers to any medium that is capable of storing, encoding or carrying a set of instructions 220 for execution by the computing device 200 and that cause the computing device 200 to perform any one or more of the methodologies of the present disclosure.

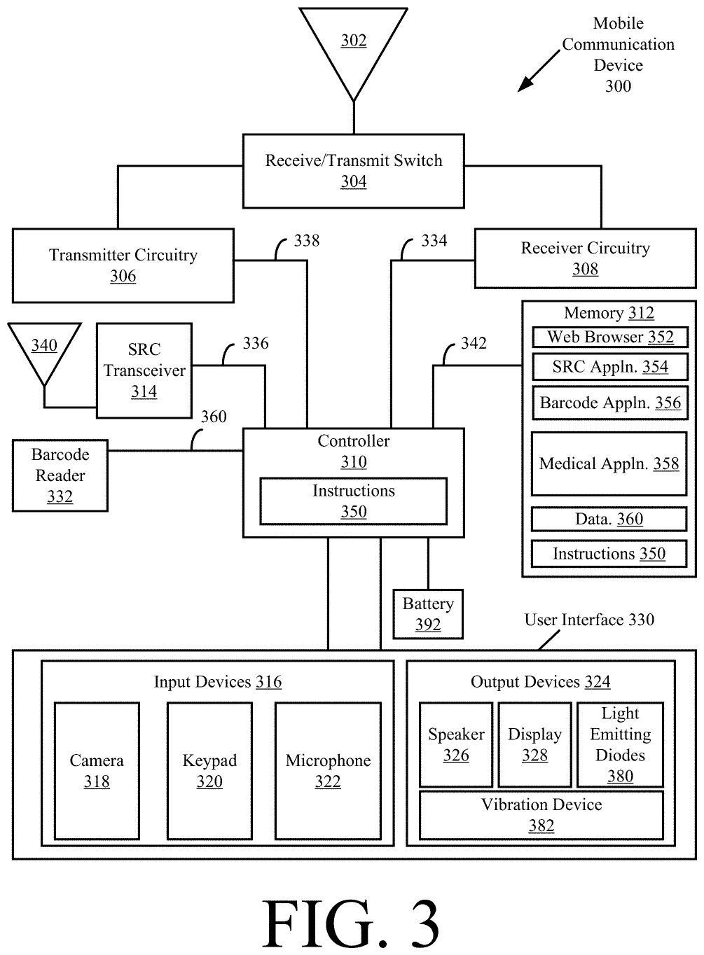

Referring now to FIG. 3, there is provided an illustration of an illustrative architecture for a mobile communication device 300. Computing device 106 of FIG. 1 may include a mobile communication device. In this scenario, computing device 106 is the same as or similar to mobile communication device 300. As such, the following discussion of mobile communication device 300 is sufficient for understanding computing device 106 of FIG. 1. Notably, the computing devices 102, 104 are shown in FIG. 1 as comprising personal computers. The present solution is not limited in this regard. Computing devices 102, 104 can alternatively comprise mobile communication devices that are the same as or similar to mobile communication device 300.

Mobile communication device 300 may include more or less components than those shown in FIG. 3. However, the components shown are sufficient to disclose an illustrative embodiment implementing the present solution. Some or all of the components of the mobile communication device 300 can be implemented in hardware, software and/or a combination of hardware and software. The hardware includes, but is not limited to, one or more electronic circuits. The electronic circuits can include, but are not limited to, passive components (e.g., resistors and capacitors) and/or active components (e.g., amplifiers and/or microprocessors). The passive and/or active components can be adapted to, arranged to and/or programmed to perform one or more of the methodologies, procedures, or functions described herein.

The mobile communication device 300 can include, but is not limited to, a notebook computer, a personal digital assistant, a cellular phone, a wearable wrist worn device, or a mobile phone with smart device functionality (e.g., a Smartphone). In this regard, the mobile communication device 300 comprises an antenna 302 for receiving and transmitting Radio Frequency (“RF”) signals. A receive/transmit (“Rx/Tx”) switch 304 selectively couples the antenna 202 to the transmitter circuitry 306 and the receiver circuitry 308 in a manner familiar to those skilled in the art. The receiver circuitry 308 demodulates and decodes the RF signals received from an external device. The receiver circuitry 308 is coupled to a controller (or microprocessor) 310 via an electrical connection 334. The receiver circuitry 308 provides the decoded signal information to the controller 310. The controller 310 uses the decoded RF signal information in accordance with the function(s) of the mobile communication device 300. The controller 310 also provides information to the transmitter circuitry 306 for encoding and modulating information into RF signals. Accordingly, the controller 310 is coupled to the transmitter circuitry 306 via an electrical connection 338. The transmitter circuitry 306 communicates the RF signals to the antenna 302 for transmission to an external device via the Rx/Tx switch 304.

The mobile communication device 300 also comprises an antenna 340 coupled to a Short Range Communications (“SRC”) transceiver 314 for receiving SRC signals. SRC transceivers are well known in the art, and therefore will not be described in detail herein. However, it should be understood that the SRC transceiver 314 processes the SRC signals to extract information therefrom. The SRC transceiver 314 may process the SRC signals in a manner defined by the SRC application 354 installed on the mobile communication device 300. The SRC application 354 can include, but is not limited to, a Commercial Off the Shelf (“COTS”) application (e.g., a Bluetooth application). The SRC transceiver 314 is coupled to the controller 310 via an electrical connection 336. The controller uses the extracted information in accordance with the function(s) of the mobile communication device 300.

The controller 310 may store received and extracted information in memory 312 of the mobile communication device 300. Accordingly, the memory 312 is connected to and accessible by the controller 310 through electrical connection 342. The memory 312 may be a volatile memory and/or a non-volatile memory. For example, memory 312 can include, but is not limited to, a RAM, a Dynamic RAM (“DRAM”), a Read Only Memory (“ROM”) and a flash memory. The memory 312 may also comprise unsecure memory and/or secure memory. The memory 312 can be used to store various other types of data 360 therein, such as authentication information, cryptographic information, location information, and various medical related information.

The mobile communication device 300 also may comprise a barcode reader 332. Barcode readers are well known in the art, and therefore will not be described herein. However, it should be understood that the barcode reader 332 is generally configured to scan a barcode and process the scanned barcode to extract information therefrom. The barcode reader 332 may process the barcode in a manner defined by the barcode application 356 installed on the mobile communication device 300. Additionally, the barcode scanning application can use camera 318 to capture the barcode image for processing. The barcode application 356 can include, but is not limited to, a COTS application. The barcode reader 332 provides the extracted information to the controller 310. As such, the barcode reader 332 is coupled to the controller 310 via an electrical connection 360. The controller 310 uses the extracted information in accordance with the function(s) of the mobile communication device 300.

As shown in FIG. 3, one or more sets of instructions 350 are stored in memory 312. The instructions may include customizable instructions and non-customizable instructions. The instructions 350 can also reside, completely or at least partially, within the controller 310 during execution thereof by mobile communication device 300. In this regard, the memory 312 and the controller 310 can constitute machine-readable media. The term “machine-readable media”, as used herein, refers to a single medium or multiple media that stores one or more sets of instructions 350. The term “machine-readable media”, as used here, also refers to any medium that is capable of storing, encoding or carrying the set of instructions 350 for execution by the mobile communication device 300 and that causes the mobile communication device 300 to perform one or more of the methodologies of the present disclosure.

The controller 310 is also connected to a user interface 330. The user interface 330 comprises input devices 316, output devices 324 and software routines (not shown in FIG. 3) configured to allow a user to interact with and control software applications (e.g., software applications 352-358 and other software applications) installed on the mobile communication device 300. Such input and output devices may include, but are not limited to, a display 328, a speaker 326, a keypad 320, a directional pad (not shown in FIG. 3), a directional knob (not shown in FIG. 3), a microphone 322, and a camera 318. The display 328 may be designed to accept touch screen inputs. As such, user interface 330 can facilitate a user software interaction for launching applications (e.g., software applications 352-358 and other software applications) installed on the mobile communication device 300. The user interface 330 can facilitate a user-software interactive session for: initiating communications with an external device; and/or writing data to and reading data from memory 312.

The display 328, keypad 320, directional pad (not shown in FIG. 3) and directional knob (not shown in FIG. 3) can collectively provide a user with a means to initiate one or more software applications or functions of the mobile communication device 300. The application software 352-358 can facilitate the data exchange (a) a user and the mobile communication device 300, and/or (b) the mobile communication device 300 and a remote computing device 102, 104, 114. In this regard, the application software 352-358 performs one or more of the following: verify the identity of a user of mobile communication device 300 via an authentication process; present information to the user indicating this his/her identity has or has not been verified; and/or present a Graphical User Interface (“GUI”) to the user for enabling the user to register with system 100, input information into system 100, receive information from system 100, and/or output the received information for encouraging the user to follow a treatment plan and/or causing the user to take actions recommended by medical professional(s).

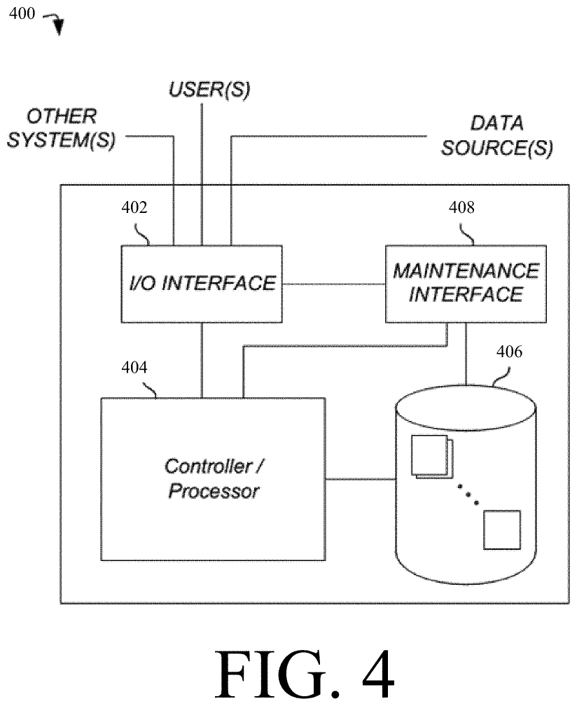

Another illustrative system 400 is provided in FIG. 4. As shown in FIG. 4, system 400 comprises a generalized computer system. System 400 includes program software executed on one or more computerized devices, and is configured to execute and access the programs and function implementing the present solution. Multiple modules, systems, units and/or sensors that may be included within system 400.

System 400 includes an I/O interface 402, a processor 404, a database 406, and a maintenance interface 408. System 400 can include one or more computers that include one or more processors and memories configured for performing tasks described herein below. This can include, for example, a computer having a CPU and non-volatile memory that stores software instructions for instructing the CPU to perform at least some of the tasks described herein. This can also include, for example, two or more computers that are in communication via a computer network, where one or more of the computers includes a CPU and non-volatile memory, and one or more of the computer's non-volatile memory stores software instructions for instructing any of the CPU(s) to perform any of the tasks described herein. Thus, while system 400 is described in terms of a discrete machine, it should be appreciated that this description is non-limiting, and that the present description applies equally to numerous other arrangements involving one or more machines performing tasks distributed in any way among the one or more machines. It should also be appreciated that such machines need not be dedicated to performing tasks described herein, but instead can be multi-purpose machines, for example computer workstations, that are suitable for also performing other tasks. Furthermore, the computers may use transitory and non-transitory forms of computer-readable media. Non-transitory computer-readable media is to be interpreted to comprise all computer-readable media, with the sole exception of being a transitory, propagating signal.

The I/O interface 402 provides a communication link between external users, systems, and data sources and components of the system 400. The I/O interface 402 is in communication with the processor 404 and database 406, and is configured to provide an interactive link between system 400 and any remote third party. The I/O interface 402 is configured to allow user(s) to input information into the system 400 via any known input device (e.g., a keyboard, a mouse, a touch screen, and/or a microphone). The I/O interface 402 provides a display portal defining a plurality of visually perceptible elements corresponding to the described functions. The I/O interface 402 is configured to allow user(s) to receive information output from the system 400 via any known output device (e.g., a display monitor, a printer, a vibration device and/or a speaker). The I/O interface 402 can be configured to allow other systems to communicate with the system 400. For example, the I/O interface 402 can allow one or more remote computer(s) to access information, input information, and/or remotely instruct the system 400 to perform one or more of the tasks described herein. The I/O interface 402 is configured to allow communication with one or more remote data sources. For example, the I/O interface 402 can allow one or more remote data source(s) to access information, input information, and/or remotely instruct the system 400 to perform one or more of the tasks described herein.

The datastore 406 provides persistent data storage (computer readable storage media, i.e. hardware) for system 400. Datastore 406 is in communication with processor 404 and I/O interface 402. Datastore 406 can be integral to or separate from the system 400, and can operate on one or more computing devices. Datastore 406 can provide non-volatile data storage for any information suitable to support the operation of the system 400, including various types of data necessary to perform the functions and feature discussed below.

Maintenance interface 408 is configured to allow user(s) to maintain a desired operation of system 400. In some scenarios, the maintenance interface 408 is configured to allow for reviewing and/or revising of data stored in the datastore 406 and/or performing any suitable administrative tasks commonly associated with database management. This can include, for example, updating database management software, revising security settings, and/or performing data backup operations. In those or other scenarios, the maintenance interface 408 is configured to allow for maintenance of the processor 404 and/or the I/O interface 402. This can include, for example, software updates and/or administrative tasks such as security management and/or adjustment of certain tolerance settings.

Processor 404 is configured to perform a process or a plurality of processes such as the processes described herein. Additionally, processor 404 includes software programmed to facilitate communications between interested parties and/or devices of the interested parties. Processor 404 includes a non-transitory computer-readable medium with instructions stored thereon to execute predetermined steps.

System 400 includes one or more software programs fully functional through corresponding hardware to enact one or more functions and key elements within system 400. System 400 is fully customizable to any area of practice, including medical for example, and works to increase uniformity of procedures when handling unusual or unique situations that arise. By having a system set to handle the unusual or unique situations which arise, the quality of care is increased and the rushed or crisis-oriented care problems are reduced. Through system 400, medical professionals are able to set goals for themselves and/or for patients. These goals may pertain to symptoms of the patient, selected conditions of the patient, and so forth.

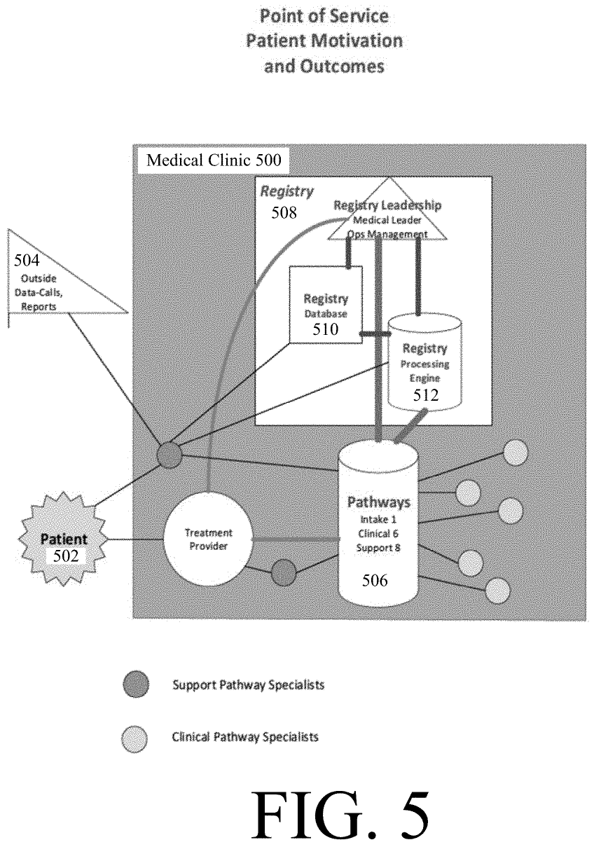

Referring now FIG. 5, there is provided an illustration that is useful for understanding operations of systems 100, 400. Different parties may use systems 100, 400. These parties include, but are not limited to, a medical clinic 500, patient(s) 502 and/or third party entities 504 (e.g., call centers, laboratories, hospitals, nursing homes, and/or data providers). The medical clinic can have staff, doctors and various software programs that are used to govern the medical clinic. Systems 100, 400 are configured to regulate the interaction and processing of data through all these parties 500, 502, 504.

There are a number of pathways 506 defined through systems 100, 400. A pathway is a set of steps or logic that is located within system to handle a particular condition or task. For example, there may be 15 pathways defined in a system 100, 400. One pathway may be for intake-onboarding of new patients. Six pathways may be for clinical actions based upon data received from the patient 502 during a visit to the medical clinic 500. Eight pathways may be for clinical support that is geared toward the handling of data and general procedures for support staff. The present solution is not limited to the particulars of this example. Any number of pathways may exist. Pathways 506 comprise a set of instructions or logic that sequentially walk through all tasks required to support the team-agreed upon actions for good outcomes. Pathways 506 may be modified as needed to fit the practice by the medical clinic 500.

The pathway-based work sequence may be displayed on an interactive electronic device (e.g., computing device 102, 104 and/or 106 of FIG. 1). The present solution may provide and track tasks for an individual such as support staff, the medical professional and/or the patient. The tracked tasks are shown in one or more pathway-based work sequences. Once a given task is completed, system 100, 400 moves to a next task in the work sequence and/or stores task related data in a data store (e.g., data store 116 of FIG. 1) that may be used later for various purposes (e.g., to modify a pathway-based work sequence for optimizing patient treatment).

Pathways 506 may be used for training, and may be selectively applied to people as necessary. One or more team leaders may be assigned to a given pathway-based work sequence, and thus be given permissions to access and modify the same in system 100, 400. The team leader may a staff who conducts workshops with individuals who have minimal educational credentials but find that they may gain benefit by using system 100, 400. The team leader or an administrator of system 100, 400 may customize employee workload(s) and work balance(s) by clustering pathways 506 together.

System 100, 400 may automatically track work performance by employees of the medical clinic 500, determine performance based metrics, and use the tracked performance information and/or performance based metrics to train a machine learning algorithm and/or to machine learn employee aptitudes (e.g., transparently). The machine learning could then be used to generate and/or modify a pathway-based work sequence that is optimized for a given employee's aptitude, and/or cause select information to be provided to the given employee that would help train and/or motivate the employee to improve performance (e.g., input information into the system 100, 400 in a more timely manner, due to the near-self-training aspect of checklist actions).

A goal of the present solution is the preparation for or anticipation of upcoming actions by medical professionals and patients. By being preemptive and prepared, no longer is the medical provider stuck in a patient-chasing or after-the-fact reactionary method as seen with most clinical operations. Medical providers and patients are empowered due to the orchestrated tools (i.e. pathways) at the point of service. An order sheet or activity flow sheet may be associated with each pathway. The order sheet or activity flow sheet is used to capture information and guide the actions of the medical provider or support personnel. This helps to maintain complete transparency in the medical clinic 500. This may also allow for reduced operational staff thereby further decreasing overhead expenses.

As noted previously, a set of best practices or predefined steps are encouraged for use by system 100, 400 to streamline and enhance the quality of patient care. Critical best practice solutions are vetted in an orchestrated team conversation of purpose. Adoption by all members of the team is sought. Actionable assessments may be implemented with professionals/staff. This may include, but is not limited to, mapping roles, organization flows, and discussion related to team outcomes that are desired. It is important to know where the medical practice is at and then to make changes. Each person in the team (e.g., medical clinic 500) can voice opinion with the hopes to aid in full participation and streamlining the pathways 506.

The clinical pathway model of checklist/sequential operational and clinical pathways support best practice fidelity. It is noted that the buy-in or adoption of the entire team into the set procedures of the pathways is key. Each person should have a voice to express ideas and concerns. System 100, 400 is configured to enable idea sharing and conversation of interested parties in the clinic. The sequential step configuration of clinical pathways promotes clinician operational staff fidelity to the pathways 506. For example, if an operational or clinical stakeholder feels that an exception to any action exists, an immediate filing of an “EXCEPTION” alert occurs for team huddle with the team leader. The present solution is not limited to the particulars of this example.

A registry 508 is provided in system 100, 400. Registry 508 includes software, processing engine(s) 512, and data store(s) 510. During operations, data is sent to the registry 508 and processed by the processing engine(s) 512. Rules governing the analyzing and handling of data are applied by the processing engine(s) 512. Patient data, whether gathered during a visit to the medical clinic 500 or by outside entities (e.g., call centers, laboratories, or other medical offices), automatically flows into the data store 510. The processing engine(s) 512 accesses the data stored in the data store 510 and uses the accessed data to generated and/or modify clinical pathways 506. The data stored in the data store 510 may include, but is not limited to, lab values, clinical assessment tool scores, and/or other data which indicate varying degrees of clinical concern or good health. The codified logic of the registry owner in interpretation of agreed upon best practices is used to sort, grade and/or script the various pathways 506 designed to enforce actions based on the data. In this way, the processed data becomes the building block of clinical pathways 506.

A lead physician (or other registry leader) may be in control of both operational and clinical activities in system 100, 400. Clear urgency conduits called “Codes” from line staff and providers to the registry leader are engineered to deliver advisement in the right way, at the right time. All hands on-deck situations where quick action improves outcomes are designated Codes Blue and Red. Gradations of urgency involve the registry leader in a way pre-programmed and anticipated by the leader. Some issues are solved by standing orders and rules. Design allows the registry leader to have an on-demand view of work occurring in the clinical or clinical support pathways 506, sensor data associated with patients (e.g., blood pressures and/or heart rates detected/measured by the wearable sensor/device 124 of FIG. 1), and/or reminders/stimulus provided to patients (e.g., via a computing device 106 of FIG. 1 and/or the wearable sensor/device 124 of FIG. 1).

As noted previously, clinical care pathways 506 are defined and managed via the registry process, rules and/or leadership actions. Quality is enhanced by this alone in patients who do not have escalated code alerts during their treatment. Clinical care pathways sequence the operational and/or clinical action-steps needed to most reliably create clinical outcomes. The action-steps enacted by the assigned operational specialist bring materials proximal to the treatment provider-patient visit event. Follow up coordination and communication post-visit are likewise clinical pathway-driven events. System 100, 400 provides point of service tools or cognitive-behavioral tools to the treatment provider.

It is also noted that one or more modules may be integrated within system 100, 400 to match the needs of staff and facilitate integration with legacy EMR systems. For example, system 100, 400 may integrate with legacy schedulers. Other functions may include, but is not limited to, note storage, prescribing an event, billing, and/or record storage. System 100, 400 is fully customizable to the particular practice of the professional.

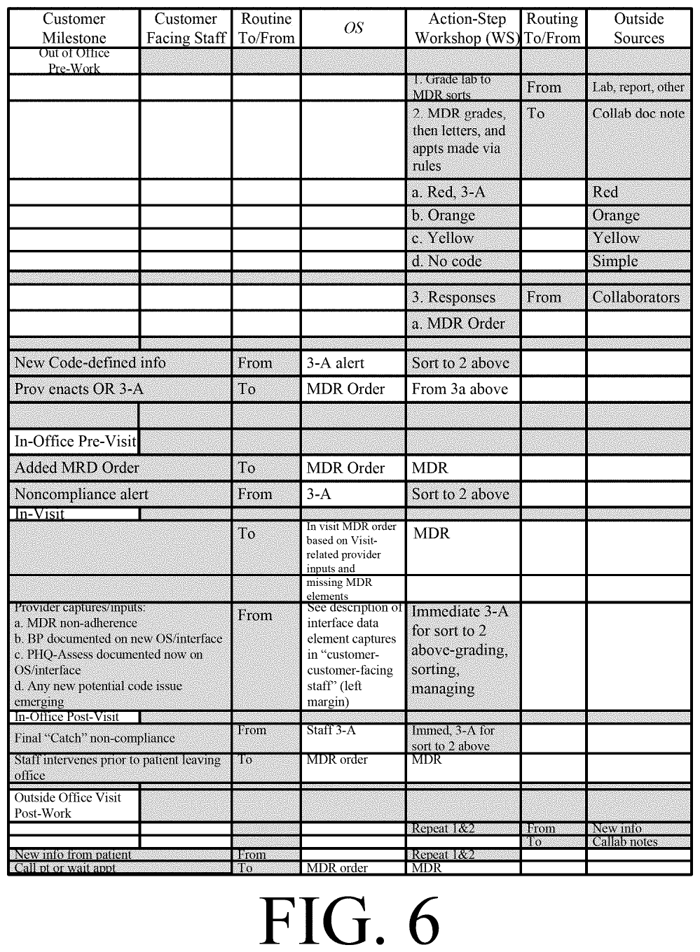

Referring now to FIG. 6, there is provided an illustration that is useful for understanding a workshop flow analysis for code processing, escalation, order sheet display, and loop closure that is implemented by system 100, 400. The programs used within system 100, 400 are built around the cyclical and repetitive nature of visits done by a patient to a medical professional. System 100, 400 is used to sequentially monitor and facilitate prescribed actions by the staff and professionals with each visit. Any number of action workshops may be attached to any number of steps within the visit process. These may apply to either or both of the staff/professional and the patient. Various worksheets to guide the actions of those in the workshop may be used to map variations therein.

FIG. 6 illustrates how input sources (via any of the patient, staff, professionals or others) interacts with the order sheet display, workshops and next action steps in the visits. The milestones or position within the treatment is listed. This can be a first visit, follow up visit, lab and so forth. The information from the customer/staff is then routed as shown to the order sheet and then the appropriate action step/workshop is then taken. From there it is routed to potential outside sources as needed.

As information is gathered in a transaction with a patient, certain grades of acuity are ascertained. This may be from any number of sources of information such as:
patient history with respect to any new significant metabolic illness, suicidal thoughts/plans and the like;
labs pertaining to significant metabolic issues like the Liver, Thyroid, Kidney, WBC or RBC issues;
observations related to a patient being late, patient delays of appointments, etc.; and
reports such as psychological testing results, collaborative notes or call with medical provider who is co-managing the metabolic or other medical conditions with shared patient.

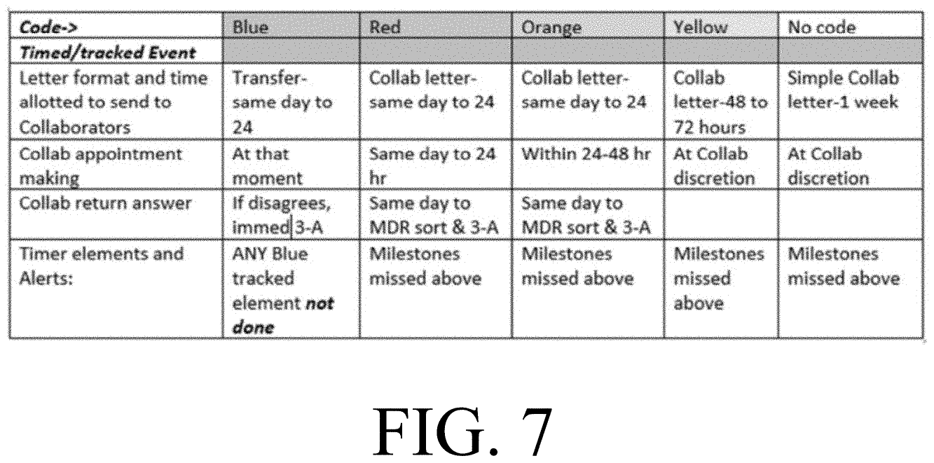

The chart shown in FIG. 7 illustrates grading rules for a medical director's review. Scores are based upon any number of factors such as the condition of the patient and their actions as noted above. The graded score can be labeled in any manner desired. These drive not only proximal orders, but may also drive provider scripting and patient motivational advisements along with education about the code target. Health empowerment and behavioral activation using cognitive-behavioral techniques (for provider and patient) is the outcome goal.

A color scale is provided herein as an example of one such method. It is important to note that system 100, 400 can be configured to monitor actions and conditions of patients to automatically generate a particular grade so as to more readily catch and escalate important processes to ensure elevated levels of care. In FIG. 7, the graded codes are colored. Each graded score (Blue, Red, Orange, Yellow, No) is matched with a specific condition to produce a proactive (delivered in-visit) actions and motivations. The provider and patient have salient motivational scripts and action plans to improve engagement and empowerment.

In action, the following may be exemplary and instructive. Consider the situation where there exists a kidney enzyme being out of range. System 100, 400 helps to facilitate the actions as follows:
script generated for the provider;
materials and actions assigned to patient;
motivational planner for how to squeeze in collaborator appointment;
generation of a treatment contract between provider and patient;
production of a cognitive time management module link in advisement packet; and
consequences of immediate attention (potentially being able to stay on medications and keep improving) versus slow or no action.

In operation, the process for the patient begins like most other visits. Data is input into the system 100, 400 as provided by the patient or collected by a staff or professional. The type of visit affects the sequence of actions and routing that may occur for the patient. As data is collected with respect to the patient, system 100, 400 continuously monitors and analyzes the data to appropriately grade or flag any issues that may be an exception to “normal” processes.

A medical director review occurs wherein a particular medical professional reviews each patient and their situation/condition. The medical director looks to isolate exceptions and progress of each patient. Codes may be assigned upon review by the medical director or automatically initiated by system 100, 400. Grading follows in accordance with particular rule sets that are customizable to each practice. FIG. 7 shows examples of such rules. Where necessary, the medical director can coordinate with other providers and staff for action plans and facilitate immediate responses where necessary. This pulls multiple disciplines together to fully address the needs of the patient.

A goal of the present solution is the integration of both behavioral/cultural change in patients and professionals along with the enhancements of the physical processes of medical treatment methods. Both patients and professionals have a responsibility for the overall effectiveness of care received. By encouraging a team approach between professionals and patients, a higher level of care may be achieved. System 100, 400 provides functions and features to generate better acceptance of the care prescribed and help them to buy-in to the treatment program. System 100, 400 also helps to facilitate appropriate mapped processes as it relates to a treatment and actions of the professionals. One or more software programs may be employed by system 100, 400 to permit better clarity, streamline actions, enhance integration, and define roles within the practice.

System 100, 400 may process and analyze data at every stage or step within the framework of treatment. Codes or grading may occur as the data is analyzed. For example, labs may be done on a patient and the laboratory data may be entered into system 100, 400. The system may assess the condition of the data in view of expected or normal readings and assign a code or grade therein. The grade may adjust the pathway or escalate the information to the medical reviewer. System 100, 400 helps to identify unexpected or not normal conditions, and ensure that the identified unexpected or not normal conditions are reviewed and assessed as needed.

Some benefits of the system 100, 400 is the detail to which it automates the processes of care. System 100, 400 may include one or more scripted techniques or dialogues that may be used to facilitate behavioral change in patients and staff. However, system 100, 400 is also configured to provide medical reviewers which serve as a check and balance wherein medical professionals are able to review and catch exceptions or variations from the level of care. The system also continuously monitors the data of each patient and the actions of professionals to assist in guiding and directing action.

System 100, 400 also helps to insulate or provide some level of immunity to the medical professionals. This can be seen with respect to malpractice claims but also through the collaborative nature of the system wherein it facilitates a greater ability to share knowledge across multiple disciplines. Furthermore, system 100, 400 is customizable to fit the particular nature of practice of the professional.

Once done, an area is selected for integration into system 100, 400. System 100, 400 includes more than just software programs but reaches into methods to enhance and improve functional interactions and improvements within a team based unit. The programming portion of system 100, 400 can then be utilized to customize a particular process to enhance the team efforts. Ideally the selected area is honed in on and assessed with eyes for improvement and integration. Acceptance is important at this stage as escalations occur with some structure and meaning. Thereafter, the system as a whole is set up to address the practice's unique needs. Modules, workshops, methods of reporting, management, and alerts are integrated as needed. Notifications or messages can be transmitted to patient or clinic staff/provider to encourage participation and individual development. At this time, patients may be integrated into the system to bring about the collective partnership of medical professional and patient as a team in providing high levels of care.

Illustrative Methods

The present solution implements a behavioral program for staff and medical providers to empower and teach patients. The behavioral program provides behavioral scripts directly to patients for actions being recommended. Computer empowered pathways are designated that are done via checklists interface formats to ensure 100% accuracy (or exception).

The present solution also provides a new patient personal device monitoring system that are used for behavioral management. Machine learning is employed to facilitate patient behavioral management. Although supplemental to the advisement and orders through the medical clinic, the personal tools provided by the monitoring system are designed to interact with the patient based on circumstances outside of the office/visit environment.

The present solution is more than just a management system. The present solution is a comprehensive best practice approach for patient, provider and the medical clinic. Management is punitive and rarely works. The preset solution breaks barriers of human behavior complacency and inaction in a persistently positive way. In order to escape the casual observer, the present solution uses mundane activities as lesson plans on the mindset, organizational approaches, and anxiety control in the service of healthy behavioral activation. It is not only about getting laboratory information. It is about prioritizing, executing, balancing and self-care.

The present solution provides tools at the right moment based on a sizing, scoping and prioritization methodologies. Accountability anchors are provided via electronic phone global tracking. Key activities of patients are tracked as to the commitment for their completion. Blood pressure monitoring for patients is provided by the present solution to facilitate mindfulness. Based on a measured blood pressure, the present solution guides the patient for mindfulness time outs and/or the provision of medication to block accentuated adrenalin responses.

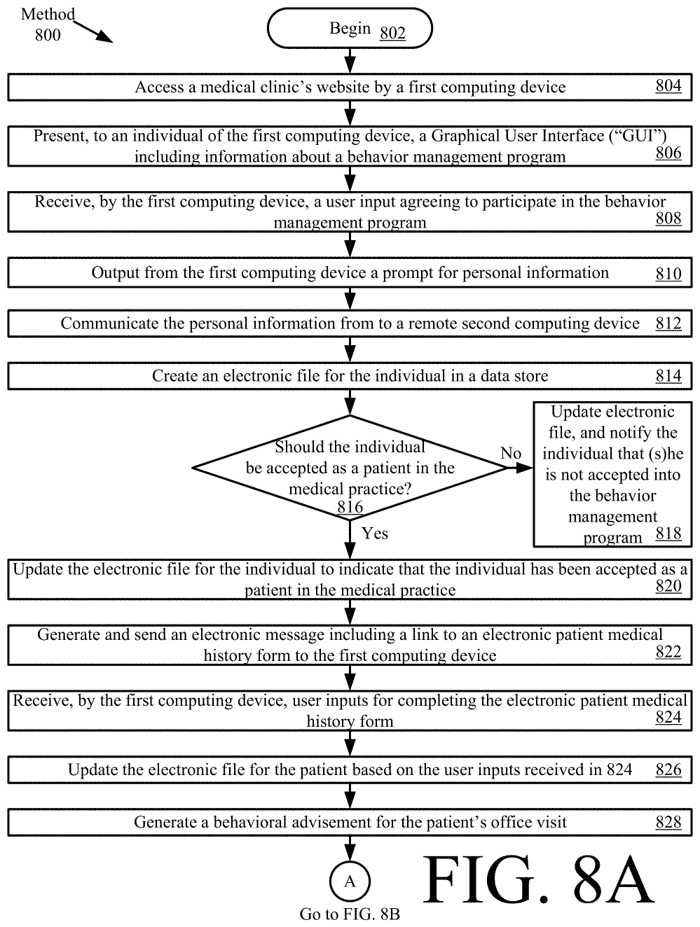

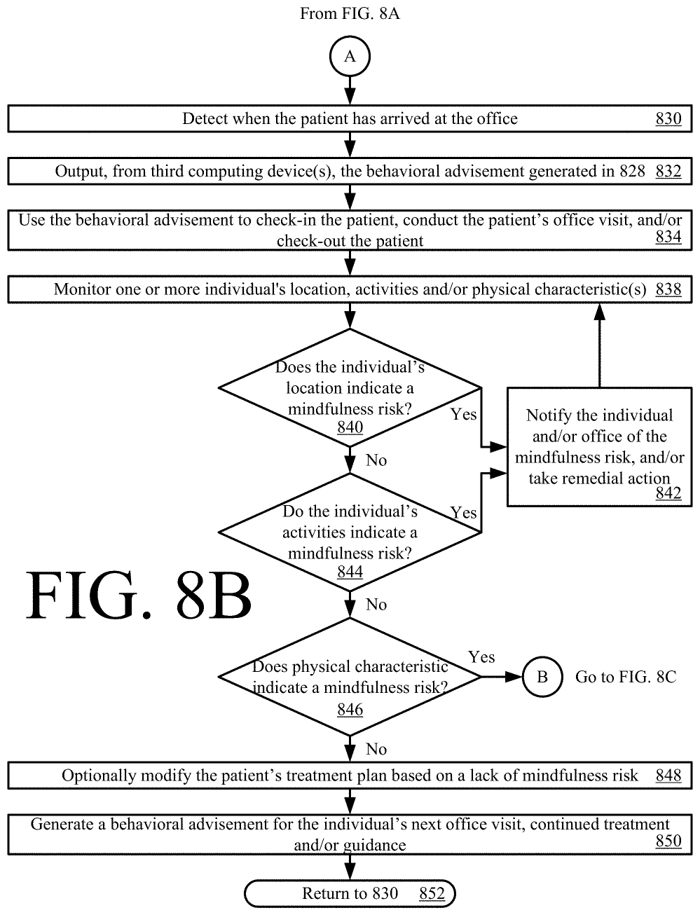

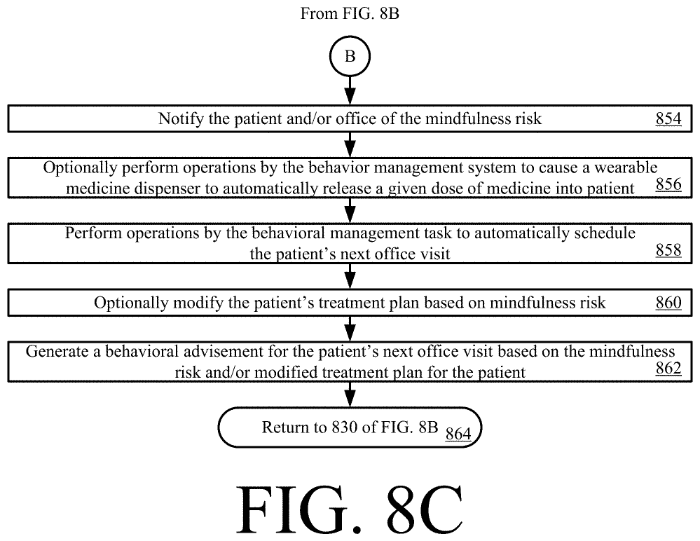

Referring now to FIG. 8, there is provided a flow diagram of an illustrative method 800 for managing a patient's behavior. Method 800 begins with 802 and continues with 804 where a website of a medical clinic (e.g., medical clinic 500 of FIG. 5) is accessed by a first computing device (e.g., computing device 106 of FIG. 1) of an individual (e.g., individual 110 of FIG. 1). The website may be hosted by a server (e.g., server 114 of FIG. 1) that is located at a facility of the medical clinic or a facility of a third party (e.g., a website host service provider). Once the website has been accessed, a GUI is presented to the individual via the first computing device, as shown by 806. The GUI includes information about a behavior management program offered by the medical clinic. In 808, the first computing device received a user input agreeing to participate in the behavior management program.

Responsive to the user input, the first computing device outputs a prompt for personal information in 810. The prompt can be presented via a form provided on a webpage of the website. The personal information can include, but is not limited to, a full name, contact information, an age, a medical diagnosis, a number of previous hospitalization, and/or substance abuse). The personal information is sent from the first computing device to a second computing device (e.g., server 114 of FIG. 1). At the second computing device, an electronic file for the individual is created and stored in a data store (e.g., data store 116 of FIG. 1).

In 816, the personal information is processed by the second computing device to determine whether the individual should be accepted as a patient in the medical practice. This decision is based on pre-defined criteria. The pre-defined criteria includes, but is not limited to, a type of medical diagnosis (e.g., depression), a level of current substance abuse (e.g., currently no substance abuse), and/or a predicted ability to perform rigorous behavior activation in a medical context for treatment. For example, the individual is not accepted into the medical practice when (s)he has a medical or psychiatric complexity that would not allow him(her) to reach the level of self-awareness and empowerment that are the targets of the behavior management program. In contrast, the individual is accepted into the medical practice when (s)he has demonstrated that (s)he has the time and dedication to complete assignments, complete exercises, and attend consultations with medical professionals. Individuals with anxiety, depression and ADHD may be accepted into the medical practice. Medications are not the entirety of the treatment equation of the behavior management program.

If the individual should not be accepted into the medical practice [816:NO], then 818 is performed where the electronic file is updated to indicate the same. 818 also involves automatically performing operations by the second computing device to notify the individual that (s)he has not been accepted into the behavior medical program. This notification can include, but is not limited to, an audio message (e.g., a pre-recorded phone message) and/or an electronic message (e.g., a text message or electronic mail message).

If the individual should be accepted into the medical practice [816:YES], then 820 is performed where the electronic file for the individual is updated by the second computing device to indicate the same. Next in 822, the second computing device generates and sends an electronic message (e.g., a text massage or electronic mail message) to the first computing device. The electronic message includes a link to an electronic patient medical history form that is to be completed by the individual. In 824, user inputs for completing the electronic patient medical history form are received by the first computing device. The completed electronic patient medical history form is then sent from the first computing device to the second computing device. The second computing device performs operations in 826 to update the electronic file of the individual.

The second computing device also generates a behavior advisement for the patient's office visit, as shown by 828. The behavior advisement can include, but is not limited to, language to motivate the patient to perform behavior management activities for improving medical condition(s), identification of area(s) that require pre-emptive planning, personal approach(es) for the patient to move stepwise behaviorally through a best-practice program for given medical condition(s), and/or action(s) that need to be performed by the patient (e.g., lab tests, family involvement, and/or cognitive exercises). Upon completing 828, 800 continues with 830 of FIG. 8B.

As shown in FIG. 8B, 830 involves detecting when a patient arrives at an office of the medical clinic. The office can be a physical office or a virtual office. In physical office visit scenarios, this detection can be made based on information input into a third computing device (e.g., computing device 102 or 104 of FIG. 1) by an employee of the medical clinic. In virtual office visit scenarios, this detection can be made based on the individual joining a virtual meeting session (e.g., a WebEx virtual meeting, a Skype virtual meeting, etc.) using the first computing device (or another computing device in the patient's possession). In 832, the third computing device (or a fourth computing device) outputs the behavioral advisement generated in 828 of FIG. 8A. The behavioral advisement is used in 834 to check-in the patient to the office, conduct the patient's office visit, and/or check-out the patient from the office. Notably, the behavioral advisement is also used to provide support, consistency and motivation to the medical providers and staff such that they are appropriately empowered and have ways to escalate problems in an optimal and efficient manner. The medical providers and staff can have greater enjoyment and personal fulfillment with their jobs when using the behavioral advisements and other tools of the behavior management system.

In some scenarios, the patient check-in process is orchestrated to begin the experience of behavioral engagement. Relationship expectations begin with the non-clinical staff of the medical clinic. The non-clinical staff use the behavior management program to interface with electronic checklists, access best language to use for interacting with the patient, and/or access a best approach for checking-in the patient. Any variance from the agreed transaction is escalated to management to apply behavioral growth-oriented solutions and contracting.

In those or other scenarios, the treatment provider begins the patient's office visit in-person or via a telemedicine connection. The patient's medical history is reviewed and/or updated. The behavior management program generates behavior activation prompts based on the patient's medical history and generates post-visit actions (e.g., labs, tests, etc.) to allow the medical provider to have the best verbiage at hand during the office visit discussion. This information is output visually from the third computing device in the possession of the medical provider. Any history that indicates significant risk is designated on the computer interface of the third computing device. The medical provider may be prompted for further information to prevent gaps and/or omission of relevant information for the medical treatment of the patient. The medical provider may perform user-software interactions with the third computing device to immediately set in motion higher levels of care commensurate for risk. For example, the medical provider can select a code type (e.g., Code Blue for requiring the patient to go to the hospital or Code Red for initiating immediate medical collaboration to address risk finding).

Referring again to FIG. 8B, method 800 continues with 838 where the patient's location, activities and/or physical characteristics are monitored by the behavior management system. Such patient monitoring may be achieved using the first computing device, a mobile phone, a smart phone, wearable sensors (e.g., a camera coupled to, in the possession of or in proximity to the patient, or a temperature sensor adhered to the individual) and/or other wearable devices (e.g., a smart watch or wrist band with integrated sensors (e.g., a blood pressure sensor, a heart rate sensor and/or a GPS sensor)). The wearable sensors/devices (e.g., wearable sensor/device 124 of FIG. 1) can sense physical movements/motions (e.g., gestures, seizures, etc.), heart rate, temperature, blood pressure, breathing patterns, and/or sleep patterns. Location information may be wirelessly communicated from the first computing device, mobile phone, smart phone and/or wireless sensor/device to the second computing device (e.g., server 114 of FIG. 1) via a network (e.g., network 112 of FIG. 1). In some scenarios, the wireless sensor/device comprises a fitness tracker (such as the Fitbit) or health monitor. Sensor data may be wirelessly communicated from the wearable sensors/devices to the second computing device (e.g., server 114 of FIG. 1) via the network (e.g., network 112 of FIG. 1). The location information and sensor data is processed and stored by the second computing device to detect whether a mindfulness risk exists in relation to the given patient. Thus, method 800 continues with determinations 840-846. According to various embodiments, when a mindfulness risk is determined to exist in relation to the given patient, whether via the location information or through other means, the computing device causes a mindfulness risk remediation event to occur.

In 840, a determination is made as to whether the patient's location indicates a mindfulness risk. For example, if the patient is a recovering alcoholic and the patient is located at a bar, then the system will determine that a mindfulness risk exists in relation to the given patient. The present solution is not limited to the particulars of the example.

If so [840:YES], then 842 is performed where the patient and/or the medical clinic is notified of the mindfulness risk. The system may also perform operations to take one or more remedial actions to reduce or eliminate the mindfulness risk. For example, the system may notify a family member that the patient is currently in a bar. The system may also send an electronic message to the patient to motivate him(her) to leave the bar and/or perform other action(s) to reduce or eliminate the mindfulness risk. The present solution is not limited to the particulars of the example.

If not [840:NO], then 844 is performed where a determination is made as to whether the patient's activities indicate a mindfulness risk. For example, the patient is performing a high stress activity that has been machine learned by the system to likely cause an emotional outburst or episode by the patient. The present solution is not limited to the particulars of the example. 842 is performed when the second computing device determines that the patient's activities indicate a mindfulness risk [844:YES]. 846 is performed when the second computing device determines that the patient's activities do not indicate a mindfulness risk [844:NO].

In 846, the second computing device determines whether at least one physical characteristic of the patient indicates a mindfulness risk. For example, the patient's blood pressure is at a level that the system has machine learned is likely to cause anxiety/panic by the patient and/or an adrenaline surge in the patient. The present solution is not limited to the particulars of the example. If a determination is made that at least one physical characteristic of the patient does not indicate a mindfulness risk [846:NO], then 848-852 are performed. 848-852 involve: optionally modifying the patient's treatment plan based on a lack of mindfulness risk; generating a behavioral advisement for the patient's next office visit and/or continued treatment; and/or returning to 830.

If a determination is made that at least one physical characteristic of the patient indicates a mindfulness risk [846:YES], then method 800 continues with 854 of FIG. 8C. As shown in FIG. 8C, 854 involves performing operations by the behavior management system (e.g., the second computing device) to notify the patient and/or medical clinic of the mindfulness risk. Additionally or alternatively, the behavior management system can dynamically select interactive media content (e.g., electronic messages, images, videos, etc.) for the patient based on the physical characteristic which triggered the mindfulness risk. The interactive media content is then provided to the patient via an electronic device (e.g., a smart phone) in (her)his possession. The interactive media content is designed to assist the patient in changing the physical characteristic such that there is no longer the mindfulness risk (as an alternative to medication or in addition to medication). In some scenarios, method 800 would measure the physical characteristic once again to detect when the mindfulness risk has been optimally addressed. If not, then the behavior management system would contact the medical clinic, family member or other entity/individual for providing further assistance to the patient. Additionally or alternatively, the behavior management system may dynamically update the interactive media content to continue to facilitate de-escalation of the mindfulness risk based on the patient's interaction with the interactive media content and/or changes/lack of changes in the physical characteristic(s) of the patient.

Next in optional 856, the behavior management system performs operations to cause a wearable medicine dispenser (e.g., wearable medicine dispenser 122 of FIG. 1) to automatically release a given dose of medicine into the patient. For example, the second computing device sends a wireless command signal to the wearable sensor/device for dispensing a given dose of medication (e.g., via network 112 of FIG. 1 and/or computing device 106 of FIG. 1). In response to the wireless command signal, the wearable medicine dispenser dispenses the given dose of medicine into the patient. The wearable medicine dispenser may also communicate a message to the second computing device indicating that a given amount of medicine was provided to the patient at a certain day/time. The present solution is not limited to the particulars of this example. For example, the wearable device can additionally or alternatively include a tactile output device such as a vibrator and/or a low voltage electrical shock that is controlled by the behavior management system in 856. In some scenarios, the wearable medicine dispenser includes a fluid chamber filled with liquid medicine, a valve for allowing liquid medicine to flow out of the chamber, and a nozzle or syringe for dispensing liquid medicine onto the patient's skin or into the patient's bloodstream. The dispensing of medicine can be controlled by a processor of the wearable medicine dispenser. This is an important advantage of the present solution since it allows for a more personalized/customized, efficient and/or effective medication dosing for the given patient. Such automated and wireless control of the wearable medicine dispenser facilitates a more effective treatment of the patient without the need for the patient's user-software interaction with the wearable medicine dispenser (e.g., depression of a physical or virtual button of the wearable medicine dispenser).

Next in 858, the behavior management system (e.g., the second computing device) automatically schedules the patient's next office visit. The next office visit can be the same day as the mindfulness risk occurrence, the day after the mindfulness risk occurrence, or another day after the mindfulness risk occurrence. The next office visit may also comprise collaboration of multiple medical professionals inside the medical clinic, and/or collaboration with other medical professional outside of the medical clinic. The behavior management system (e.g., the second computing device) may also perform operations to: modify the patients treatment plan based on the mindfulness risk (as shown by 860); and/or generate a behavioral advisement for the patient's next office visit based on the mindfulness risk and/or the modified treatment plan (as shown by 862). Subsequently, 864 is performed where method 800 returns to 830 of FIG. 8B.

Although method 800 was discussed above in relation to monitoring the patient's location, activities and physical characteristics, the present solution is not limited in this regard. This type of monitoring applies equally to medical providers and staff. The behavior monitoring system may be configured to monitor the location, activity and physical characteristics of a medical provider and/or staff while they are at the medical clinic's facility. This monitored information is then used to optimize the person's job effectiveness, performance and satisfaction. For example, the behavior monitoring system detects an increased blood pressure level of a medical provider while (s)he is conducting an office visit for a given patient. In response to this detection, the behavior monitoring system notifies the medical provider. The medical provider can then use this information to change (her)his response to the situation and/or change the methodology used to manage the situation. The present solution is not limited to the particulars of this example.

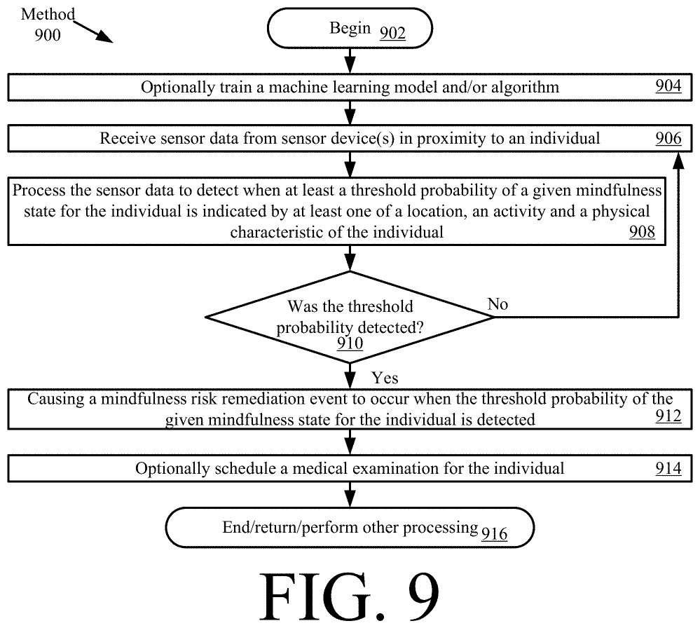

Referring now to FIG. 9, there is provided a flow diagram of an illustrative method for operating a system (e.g., system 100 of FIG. 1) to facilitate behavior management of individual(s). Method 900 begins with 902 and continues with 904 where a machine learning model and/or algorithm is optionally trained. This training can be achieved using a training dataset comprising sensor data generated by sensor device(s) associated with an individual (e.g., patient 110 of FIG. 1) and/or other individuals (e.g., other patients of the same or different medical clinic/practice). Techniques for training machine learning models/algorithms are well known.

In 906, sensor data is received from sensor device(s) (e.g., device 122 of FIG. 1) in proximity to the individual. The sensor devices can include, but are not limited to, camera(s), GPS device(s), fitness tracker(s), thermometer(s), smart phone(s), and/or health monitor(s). The sensor device(s) may be wearable sensor devices configured to be worn by the individual. The sensor device(s) may be configured to sense physical movements, object motions, heart rate, temperature, blood pressure, breathing patterns, and/or sleep patterns.

In 908, the sensor data is processed by a computing device (e.g., device 102, 104, 106, 114, 122 and/or 124 of FIG. 1) to detect when at least a threshold probability of a given mindfulness state for the individual is indicated by at least one of a location, an activity and a physical characteristic of the individual. The threshold probability can be detected, for example, when there is at least a 75% probability or likelihood that the individual currently has or will have a mental health episode (e.g., experience depression), have a particular thought or feeling, and/or perform a given harmful action. This detection can be made using the trained machine learning model and/or algorithm. Machine learning models and/or algorithms are well known.

Method 900 return to 906 when the threshold probability is not detected [910:NO]. In contrast, method 900 continues to 912 when the threshold probability is detected [910:YES]. 912 involves performing operations by the computing device to cause a mindful risk remediation event to occur. The mindfulness risk remediation event can include, but is not limited to, dispensing the given amount of medication to the individual, and/or generating and sending a notification notifying one or more parties of the threshold probability of the given mindfulness state has been determined for the individual. For example, the computing device can cause actuation of a wearable medication dispenser for dispensing a given quantity of medication into the individual.

In some scenarios, the computing device or another computing device automatically schedules a medical examination for the individual as shown by 914. Subsequently, 916 is performed where method 900 ends, returns to a previous operation and/or continues with other processing/operations.

The particular embodiments disclosed above are illustrative only, as the application may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

We claim:

1. A method for operating a system to facilitate behavior management of an individual, comprising:
receiving, by a computing device, sensor data from at least one sensor device in proximity to the individual;
processing, by the computing device, the sensor data to detect when at least a threshold probability of a given mindfulness state for the individual is indicated by at least one of a location, an activity and a physical characteristic of the individual; and
causing, by the computing device, a mindfulness risk remediation event to occur when the threshold probability of the given mindfulness state for the individual is detected.

2. The method according to claim 1, wherein the mindfulness risk remediation event includes dispensing a given quantity of medication from a medication dispenser being worn by the individual.

3. The method according to claim 1, wherein the mindfulness risk remediation event comprises generating and sending a notification to a remote device.

4. The method according to claim 1, further comprising, using a second computing device, to automatically schedule a medical examination for the individual when the threshold probability of the given mindfulness state for the individual is detected.

5. The method according to claim 1, wherein the at least one sensor device comprises at least one of a camera, a global positioning system device, a fitness tracker, a thermometer, a smart phone, and a health monitor.

6. The method according to claim 1, wherein the at least one sensor device is worn by the individual.

7. The method according to claim 1, wherein the at least one sensor device is configured to sense at least one of physical movements or motions, heart rate, temperature, blood pressure, breathing patterns, and sleep patterns.

8. The method according to claim 1, wherein the threshold probability of the given mindfulness state is detected using a machine learning model or algorithm.

9. The method according to claim 8, wherein the machine learning model or algorithm is training using sensor data associated with at least one of the individual and another individual.

10. A system, comprising:
at least one sensor device configured to collect sensor data associated with an individual; and
a computing device, including a processor and a memory, configured to store programming instructions that, when executed by the processor, cause the computing device to:
receive the sensor data from the at least one sensor device;
process the sensor data to detect when at least a threshold probability of a given mindfulness state for the individual is indicated by at least one of a location, an activity and a physical characteristic of the individual; and
cause a mindfulness risk remediation event to occur when the threshold probability of the given mindfulness state for the individual is detected.

11. The system according to claim 10, wherein the mindfulness risk remediation event comprises automatically actuating a medication dispenser for dispensing a given quantity of medication into the individual.

12. The system according to claim 11, wherein the medication dispenser comprises:
a fluid chamber filled with liquid medicine;
a valve for allowing liquid medicine to flow out of the fluid chamber; and
a nozzle or syringe for dispensing the liquid medicine onto skin of the individual or into a bloodstream of the individual.

13. The system according to claim 10, wherein the mindfulness risk remediation event comprises generating and sending a notification notifying one or more parties of the mindfulness risk.

14. The system according to claim 10, wherein the programming instructions, when executed by the processor, cause the computing device to cause an automatic scheduling of a medical examination for the individual.

15. The method according to claim 10, wherein the at least one sensor device comprises at least one of a camera, a global positioning system device, a fitness tracker, a thermometer, a smart phone and a health monitor.

16. The system according to claim 10, wherein the at least one sensor device is worn by the individual.

17. The system according to claim 10, wherein the at least one sensor device is configured to sense at least one of physical movements, object motions, heart rate, temperature, blood pressure, breathing patterns and sleep patterns.

18. The system according to claim 10, wherein a machine learning model or algorithm is used to detect the threshold probability of the given mindfulness state for the individual.

19. The system according to claim 18, wherein the machine learning model or algorithm is trained based on sensor data associated with at least one of the individual and another individual.
